(19) United States
(12) Patent Application Publication
(10) Pub. No.: US 2007/0073223 A1
(43) Pub. Date: Mar. 29, 2007

(54) Push rod of a syringe

(76) Inventor: Hung-Chi Huang, Taipei (TW)

Correspondence Address: Hung-Chi Huang, 235 Chung-Ho, Box 8-24, Taipei, TW

(21) Appl. No.: 11/175,843

(22) Filed: Jul. 6, 2005

(51) Int. Cl. A61M 5/00; A61M 5/315

(52) U.S. Cl. 604/110; 604/218

(57) ABSTRACT

A push rod of a syringe has a body; a rear end of the push rod being formed with a press portion and a front end thereof being formed with an extrusion portion. The extrusion portion comprises an axial rod extending axially and integrally from the push rod; and a front end of the axial rod being formed with a hook; a plurality of posts; a flexible unit made of rubber material; the flexible unit enclosing the plurality of posts and the axial rod, but the hook exposes out. The posts may be round posts. The outer walls of the post and the axial rod may be formed with annular recesses or flanges. Furthermore, the present invention further comprises a syringe which is installed with the push rod.

### FIELD OF THE INVENTION

The present invention relates to syringes, and in particular to a push rod of a syringe, in which the push rod has a simple structure so as to reduce the cost of the syringe. The posts and rods of the present invention is axially arranged with the same orientation of the shaft of the push rod so that the flexible unit can be drown out easily without being destroyed.

### BACKGROUND OF THE INVENTION

The prior art syringe is made of a cylinder, a push rod, and a syringe needle. The push rod pushes drug liquid from the cylinder into the human body. However to completely push the liquid into the body, a rubber plug encloses a front end of the push rod. Thereby by the deformation of the plug, the liquid can be completely pushed into the body.

In the prior art, the plug is made as a rubber sleeve which covers the front end of the push rod. In another improvement, as shown in FIG. 5, a hard plastic shaft rod is formed at the front end of the push rod. A limiting unit and a connecting unit are transversally and vertically installed to a shaft of the push rod. Furthermore, deformable rubber is injected to enclose the connecting unit and part of the shaft so as to form a rubber plug.

By this way, the assembly can be performed rapidly and the yield ratio is high. However the price is high, and this it is not suitable for the syringe since it must be updated frequently. Moreover it has a great effect to environment protection. Thereby the connecting unit is vertical to the shaft so that it induces a difficult in the mold manufacture so as to increase the cost. Furthermore, since the material includes plastics and rubber, it must take longer time as reuse treatment. Thereby in reuse process, the rubber is destroyed for separating with plastics.

### SUMMARY OF THE INVENTION

Accordingly, the object of the present invention is to improve the defect in the prior art, and thus the present invention provides a push rod of a syringe which has a simple structure so as to reduce the cost of the syringe. The posts and rods of the present invention is axially arranged with the same orientation of the shaft of the push rod so that the flexible unit can be drown out easily without being destroyed. Thus no waste material generates to achieve the object of environment protection.

To achieve above object, the present invention provides a push rod of a syringe is disclosed. The push rod has a body; a rear end of the push rod being formed with a press portion and a front end thereof being formed with an extrusion portion. The extrusion portion comprises an axial rod extending axially and integrally from the push rod; and a front end of the axial rod being formed with a hook; a plurality of posts; a flexible unit made of rubber material; the flexible unit enclosing the plurality of posts and the axial rod, but the hook exposes out. The posts may be round posts. The outer walls of the post and the axial rod may be formed with annular recesses or flanges. Furthermore, the present invention further comprise a syringe which is installed with the push rod.

The various objects and advantages of the present invention will be more readily understood from the following detailed description when read in conjunction with the appended drawing.

### BRIEF DESCRIPTION OF THE DRAWINGS

FIG. 1 is an exploded schematic view of the syringe of the present invention.

FIG. 2 is a schematic cross sectional view about the syringe of the present invention.

FIG. 3 is a partial schematic perspective view showing the frame at the push rod according to the present invention.

FIG. 4 is a partial schematic view about the extrusion portion of the present invention.

FIG. 5 is a schematic view showing the prior art structure.

### DETAILED DESCRIPTION OF THE INVENTION

In order that those skilled in the art can further understand the present invention, a description will be described in the following in details. However, these descriptions and the appended drawings are only used to cause those skilled in the art to understand the objects, features, and characteristics of the present invention, but not to be used to confine the scope and spirit of the present invention defined in the appended claims.

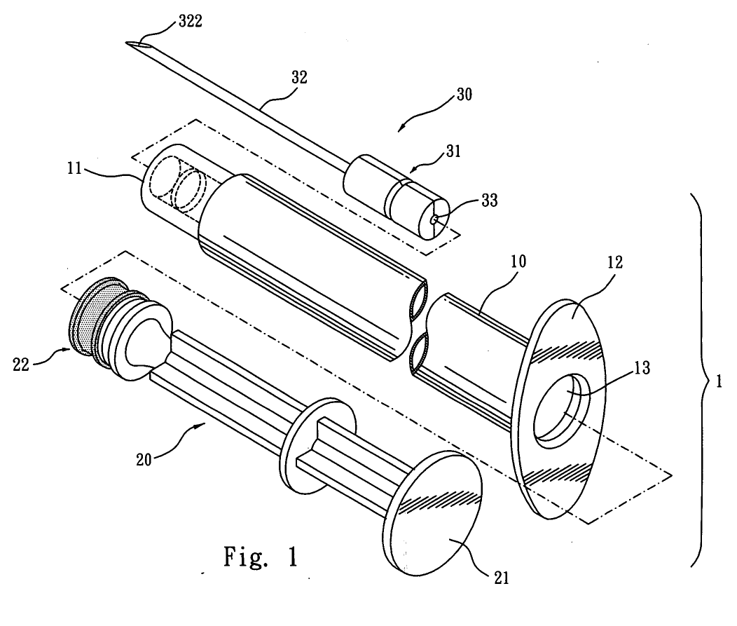

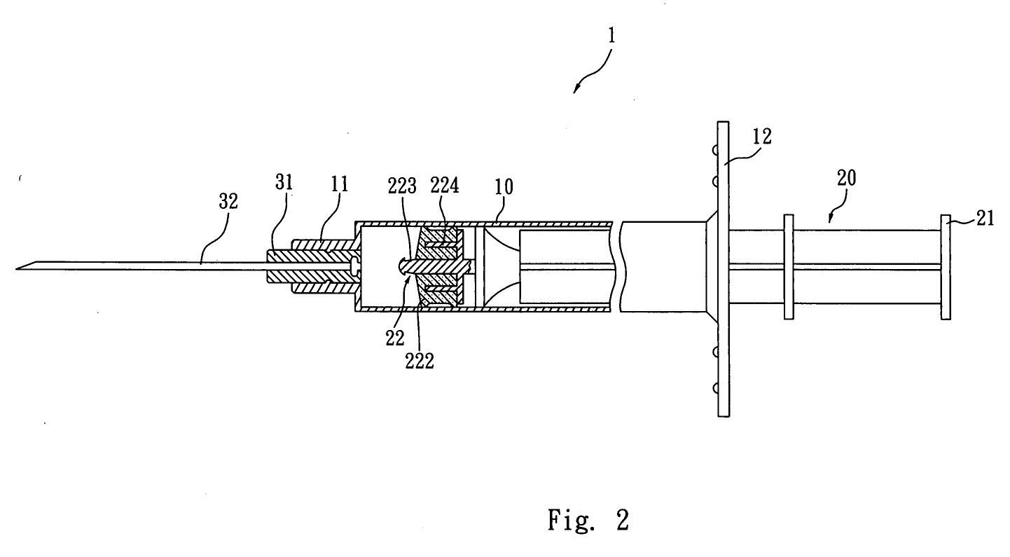

With reference to FIGS. 1 and 2, the present invention has the following elements.

A cylinder 10 is a hollow cylinder. A front end thereof is formed with a receptacle 11. A distal end of the cylinder 10 is an enlarged push portion 12. An internal of the cylinder 10 has a penetrating receiving hole 13.

A hard plastic push rod 20 is received within the receiving hole 13 from a front end opening of the cylinder 10. The push rod is capable of reciprocally movement within the receiving hole 13. A rear end of the push rod 20 is installed with a press portion 21 having a size slightly larger than that of the push rod 20 and the receiving hole 13 and a front end thereof is extended with an extrusion portion 22. A size of the extrusion portion 22 is equal to that of the receiving hole 13 so that the extrusion portion 22 is tightly resisted against an inner wall of the receiving hole 13.

A syringe needle 30 includes a needle seat 31 and a needle body 32. The needle seat 31 has a round block. The needle seat 31 is a round cylinder. A front end at an axis of the needle seat 3 1 encloses a needle body 32. The needle body 32 has an injection hole 322. A rear end of the axis of the needle seat 31 has a receiving groove 33.

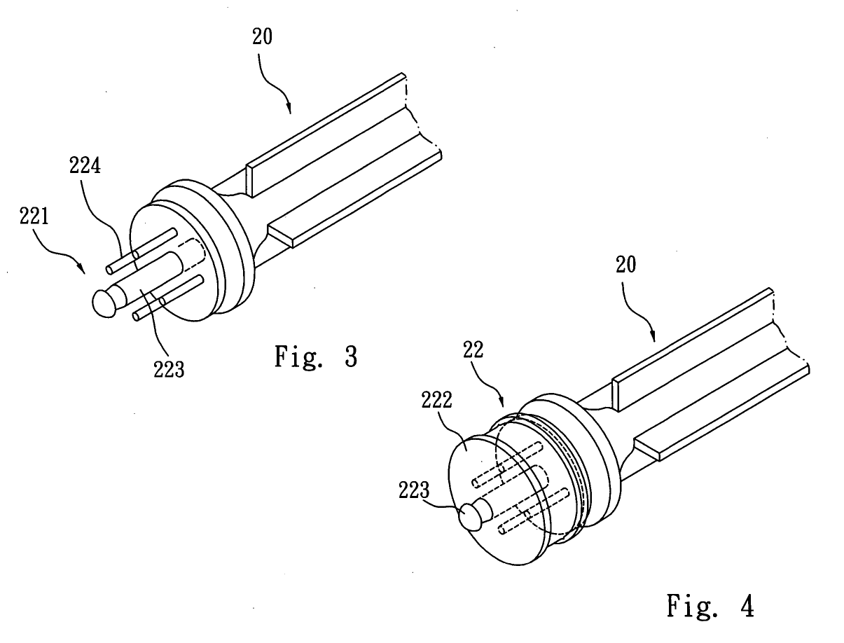

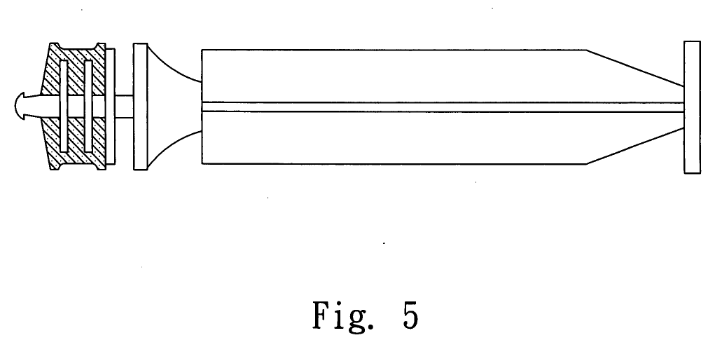

Referring to FIGS. 3 and 4, the extrusion portion 22 at the front end of the push rod 20 has a frame 221 and a flexible unit 222. A front end of an axis of the push rod 20 is extended with a shaft 223. A front end of the shaft 223 has an axial rod 223 and a plurality of round posts 224 around the axial rod 223. A front end of the axial rod 223 has a hook 2231. Outer walls of the axial rod 223 and the plurality of post 224 are enclosed by rubber material, but the hook 2231 exposes out to form the frame 221 of the extrusion portion 22. The flexible unit 222 encloses the frame 221.

In the present invention, the axial rod 223 is formed at an axial direction of the shaft 223 so that the mold for forming the push rod 20 can be formed easily and thus the cost is reduced. Furthermore, the flexible unit 222 can be pulled out since the axial rod and the posts are enclosed by rubber. Furthermore, in the present invention, by the axial rod and the posts, the frame 221 is tightly engaged to the flexible unit 222.

The outer walls of the axial rod 223 and the post 224 can be formed with annular recesses or flanges for enhancing the engagement between the frame 221 and the flexible unit 222 so that the flexible unit 222 will not fall out as the push rod 20 moves reciprocally.

Furthermore, in the present invention, the flexible unit 222 encloses the flexible unit 222 is formed by molding injecting rubber material. The operation can be performed rapidly and frequently so as to enhance the engagement of the frame to the flexible unit.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A push rod of a syringe; the push rod having a body; a rear end of the push rod being formed with a press portion and a front end thereof being formed with an extrusion portion; the extrusion portion comprising:
an axial rod extending axially and integrally from the push rod; and a front end of the axial rod being formed with a hook;
a plurality of posts;
a flexible unit made of rubber material; the flexible unit enclosing the plurality of posts and the axial rod, but the hook exposes out.

2. The push rod of a syringe as claimed in claim 1, wherein the posts are round posts.

3. The push rod of a syringe as claimed in claim 2, wherein the outer walls of the post are formed with annular recesses or flanges.

4. The push rod of a syringe as claimed in claim 2, wherein the outer wall of the axial rod is formed with an annular recess or a flange.

5. A syringe comprising:
a syringe having a receptacle at a front end thereof and a press portion at a rear end thereof; a receiving hole being formed at an interior of the syringe;
a push rod movably received in the cylinder; the extrusion portion comprising: an axial rod extending axially and integrally from the push rod; a plurality of posts; a flexible unit made of rubber material; the flexible unit enclosing the plurality of posts and the axial rod, but the hook exposes out;
a syringe needle having a needle seat and a needle body; the syringe needle is located into the receptacle of the cylinder through the needle seat.

5. The syringe as claimed in claim 6, wherein a front end of the axial rod is formed with a hook which is not enclosed by the flexible unit.

6. The syringe as claimed in claim 6, wherein an axial center of the needle seat is formed with a receiving groove.
